(19) United States
(12) Patent Application Publication
(10) Pub. No.: US 2026/0301877 A1
(43) Pub. Date: Oct. 1, 2026

(54) RECOGNITION PHYSICS-BASED SYSTEM AND METHOD FOR SIMULATING AND PREDICTING BIOLOGICAL PROCESS DYNAMICS

(71) Applicant: Jonathan Washburn, Austin, TX (US)

(72) Inventor: Jonathan Washburn, Austin, TX (US)

(21) Appl. No.: 19/630,155

(22) Filed: Mar. 26, 2026

Related U.S. Application Data
(63) Continuation-in-part of application No. 19/305,532, filed on Aug. 20, 2025.
(60) Provisional application No. 63/778,998, filed on Mar. 27, 2025.

(51) Int. Cl. G16C 20/10

(52) U.S. Cl. CPC G16C 20/10

(57) ABSTRACT

A computer-implemented system and method for simulating time-dependent biological processes involving DNA are disclosed. Using Recognition Physics (RS) and a DNA Recognition Physics state D(t)=(S(t), H(t), E(t)), the system receives an initial state and RS-based evolution rules, updates the state over time using a DNA Lagrangian, a DNA operator, and a DNA Recognition Transform, and generates predicted functional outputs. A computational architecture includes an input module, a dynamic simulation engine, and an output module, the dynamic simulation engine comprising a state manager, a rule engine, RS construct solvers, and a time stepper configured to maintain the current state, apply rules, propagate the state across simulation intervals, and generate predicted output time-series and state-variable output data. The invention supports prediction of gene regulation, drug response, circadian behaviour, environmental stress response, and DNA repair dynamics.

### CROSS-REFERENCE TO RELATED APPLICATIONS

This application is a continuation-in-part of U.S. Nonprovisional Patent Application No. Ser. No. 19/305,532 filed on Aug. 20, 2025, which claims the benefit of U.S. Provisional Patent Application No. 63/778,998, filed Mar. 27, 2025, titled “RECOGNITION PHYSICS-BASED SYSTEM AND METHOD FOR SIMULATING AND PREDICTING BIOLOGICAL PROCESS DYNAMICS”.

### FIELD OF THE INVENTION

The present invention relates generally to computational modeling of biological systems and, more particularly, to systems and methods for simulating and predicting time-dependent biological process dynamics involving DNA using a Recognition Physics-based dynamic state framework.

### BACKGROUND OF THE INVENTION

Understanding the time-dependent dynamics of biological processes, including gene regulation, cellular response to environmental stimuli, drug response, circadian behavior, and DNA repair, remains a significant challenge in computational biology. Predictive modeling of such processes is important for applications in medicine, biotechnology, and systems-level analysis of living organisms.

Conventional computational approaches typically rely on kinetic or statistical models that depend on empirically measured or fitted parameters, such as reaction rates, binding affinities, and molecular concentrations. These approaches often treat DNA as a largely static template or represent its dynamics only through simplified approximations. As a result, such models may have limited predictive capability outside the conditions for which they were calibrated and may not adequately capture integrated changes in sequence accessibility, structural conformation, and energetic or coherence state over time.

Accordingly, there remains a need for a unified, physics-based framework for simulating and predicting biological process dynamics, particularly those involving DNA, from first principles. Such a framework should support dynamic state evolution, reduce reliance on empirical parameter fitting, and provide a computational architecture capable of receiving biological state information, applying evolution rules and governing constructs over time, and generating predicted outputs for a variety of biological contexts.

### SUMMARY OF THE INVENTION

The present invention provides systems and methods for simulating and predicting time-dependent dynamics of biological processes involving DNA using a Recognition Physics (RS)-based framework. In contrast to conventional kinetic or statistical approaches that rely on empirically measured or fitted parameters, the disclosed systems and methods employ governing constructs based on RS to model biological state evolution over time.

In one aspect, the invention provides a computer-implemented method for simulating a biological process involving a DNA molecule. The method may comprise: (a) defining an initial DNA Recognition Physics (DNARP) state D(t0)=(S(t0), H(t0), E(t0)), where S represents sequence accessibility, H represents shape or structural conformation, and E represents energy or coherence; (b) defining one or more RS-based evolution rules specifying how at least one of S, H, or E changes over time in response to one or more biological interactions, conditions, perturbations, or stimuli; (c) evolving the state D(t) over a time interval by applying one or more time-dependent RS constructs, including a DNA Lagrangian LDNA(t), a DNA operator H\hat{ }DNA(t), and a DNA Recognition Transform FDNA(E(t)); and (d) generating one or more predicted functional outputs and time-dependent state trajectories.

In another aspect, the invention provides a computational system for carrying out the above method. The system may comprise: (a) an input module for receiving or defining the initial DNARP state and one or more RS-based evolution rules; (b) a dynamic simulation engine configured to evolve the state D(t) over one or more simulation intervals using the disclosed governing constructs; and (c) an output module for generating, storing, and presenting predicted output data. In some embodiments, the dynamic simulation engine may include a state manager configured to maintain a current DNARP state, a rule engine configured to evaluate and apply one or more evolution rules, one or more RS construct solvers configured to apply the governing constructs, and a time stepper configured to advance the simulation across time.

In a further aspect, the invention provides one or more non-transitory computer-readable media storing instructions that, when executed by one or more processors, cause the one or more processors to receive an initial DNARP state, receive one or more RS-based evolution rules, maintain a time-dependent biological state, propagate that state over one or more intervals using the disclosed governing constructs, and generate predicted output time-series and state-variable output data.

The disclosed systems and methods provide a computational framework for representing and simulating biological dynamics by integrating sequence accessibility, conformational behavior, and energy or coherence state in a common dynamic representation. The framework may be used in connection with gene regulation, drug response, environmental stress response, circadian behavior, DNA repair, epigenetic dynamics, and other time-dependent biological processes.

### BRIEF DESCRIPTION OF THE DRAWINGS

FIG. 1 illustrates a DNA Lagrangian stability functional.

FIG. 2 illustrates a DNA operator coherence construct.

FIG. 3 illustrates a DNA Recognition Transform.

FIG. 4 illustrates a time-dependent Schrödinger-like equation.

FIG. 5 illustrates an approximate propagation step.

FIG. 6 illustrates a time-dependent DNA Recognition Transform.

FIG. 7 illustrates a relation between a coherence state index, a base rate, and a resulting output rate.

FIG. 8 illustrates a system architecture diagram.

FIG. 9 illustrates a method flowchart for simulating and predicting biological process dynamics.

FIG. 10 illustrates a schematic of DNA dynamics as sequence accessibility, DNA shape, and coherence energy evolve over time.

FIG. 11 illustrates an example application of gene activation.

FIG. 12 illustrates an expanded computational system architecture for simulating and predicting biological process dynamics, including an input module, a dynamic simulation engine having a state manager, a rule engine, RS construct solvers, and a time stepper, and an output module configured to generate predicted output time-series and state-variable output data.

### LIST OF REFERENCE NUMERALS

100—DNA Lagrangian LDNA stability functional
102—Stability variable C(r)
104—Periodicity parameter P
106—Constant κDNA
108—Constant λDNA
110—DNA operator H\hat{ }DNA coherence construct
112—Position variable x
114—Operator term d/dx
116—DNA length constant XDNA
118—Coherence factor kDNA
120—DNA Recognition Transform FDNA(E)
122—Stabilization factor Sstab
124—Energy level En
126—Coherence constant Ecoh
128—Periodicity parameter P0
130—Time-dependent Schrödinger-like equation
132—Quantum state ψ(x, t)
134—Reduced Planck constant ℏ
136—Time-dependent DNA operator H\hat{ }DNA(t)
138—Imaginary unit i (as used in the Schrödinger-like evolution equation).
140—Approximate propagation step
142—Exponential operator e\hat{ }(−iH\hat{ }DNAΔt/ℏ)
144—Evolved state ψ(t+Δt)
150—Time-dependent DNA Recognition Transform FDNA(E(t))
152—Time-dependent stabilization factor Sstab(t)
154—Time-dependent energy level En(t)
156—Coherence constant Ecoh
158—Periodicity parameter P0
160 Coherence state index n(t)
162—Base rate R0
164—Output rate R(t)
170—System architecture
172—Input module
174—Dynamic simulation engine
176—Output module
180—Method flowchart
182—Initialization step
184—Definition of evolution rules
186—Time evolution loop
188—Output prediction
190—DNA dynamics schematic
192—Sequence accessibility S(t)
194—DNA shape H(t)
196—Coherence energy E(t)
200—Example application of gene activation
202—Activator molecule
204—DNA region
206—Coherence state E1
208—Coherence state E2
210 Expression rate
220—Expanded computational system architecture
222—Initial DNARP state and evolution rule input
224—State manager
226—Rule engine
228—RS construct solvers
230—Time stepper
232—Predicted output time-series
234—State-variable output data

### DETAILED DESCRIPTION

The invention is grounded in the principles of Recognition Physics (RS), a parameter-free framework derived from the axiom that observation and recognition interactions govern physical systems. Building upon this foundation, the DNA Recognition Physics (DNARP) model encodes the state of a DNA molecule as a triplet D=(S, H, E), where S denotes the sequence component, H denotes the shape component, and E denotes the energy or coherence component. The DNARP triplet is configured to provide a structured representation of DNA states suitable for both static and dynamic modeling.

As illustrated in FIG. 1, the DNA Lagrangian LDNA stability functional (100) is configured to govern the structural stability of DNA. The stability variable C(r) (102) evolves according to the periodicity parameter P (104) and is modulated by constants κDNA (106) and λDNA (108). This functional is operative to characterize stable conformations mathematically within RS without reliance on empirical curve-fitting, thereby providing a parameter-free description of DNA stability.

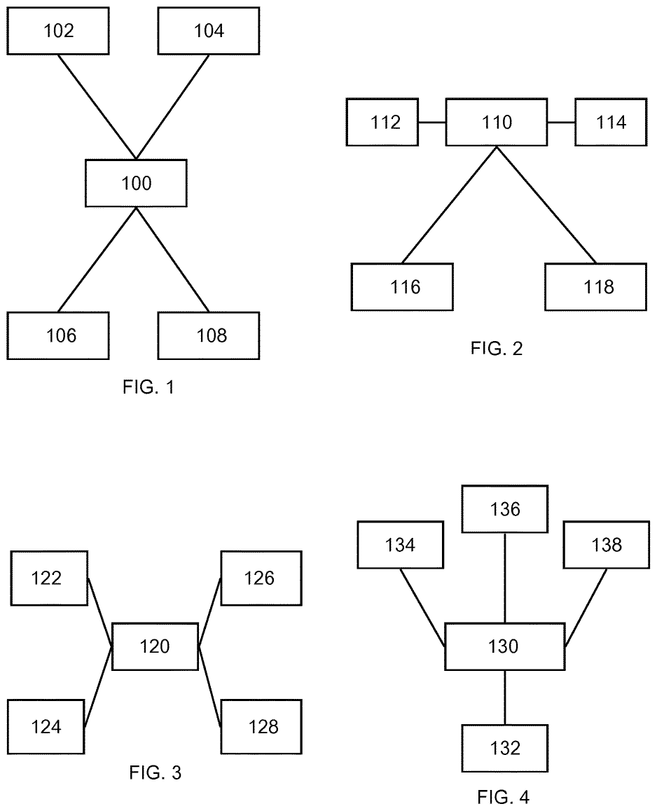

As illustrated in FIG. 2, the DNA operator H\hat{ }DNA coherence construct (110) is configured to govern the quantum-like coherence properties of DNA. The operator includes the position variable x (112) and operator term d/dx (114), and is scaled by the DNA length constant XDNA (116) and the coherence factor kDNA (118). The operator is operative to yield eigenvalues corresponding to discrete coherence levels that define quantized energy states of the DNA molecule.

As illustrated in FIG. 3, the DNA Recognition Transform FDNA(E) (120) is configured to map discrete energy levels into measurable amplitudes. The transform is determined by the stabilization factor Sstab (122), energy level En (124), coherence constant Ecoh (126), and periodicity parameter P0 (128). The transform is operative to provide predicted outputs, such as gene expression intensity or coherence state amplitude, from RS-derived energy states.

Together, the constructs LDNA (100), H\hat{ }DNA (110), and FDNA(E) (120) are configured to provide a parameter-free, static description of DNA structure, coherence, and function under the RS framework. These constructs form the foundation for extending DNARP to dynamic biological processes, as described in the following sections.

Biological processes are inherently dynamic, requiring the DNARP framework to be extended from static representations to time-dependent constructs. In the dynamic model, the DNA state is represented as D(t)=(S(t), H(t), E(t)), where each component of the triplet may evolve over time according to RS-based rules that describe interactions with proteins, signaling molecules, environmental factors, or internal feedback.

As illustrated in FIG. 4, the time-dependent Schrödinger-like equation (130) governs the evolution of the quantum state ψ(x, t) (132). The operator H\hat{ }DNA(t) (136) acts on the state, with evolution scaled by the reduced Planck constant h-bar (134). This equation defines how DNA coherence states propagate over time under the influence of interactions and external stimuli, establishing a parameter-free dynamic law of motion.

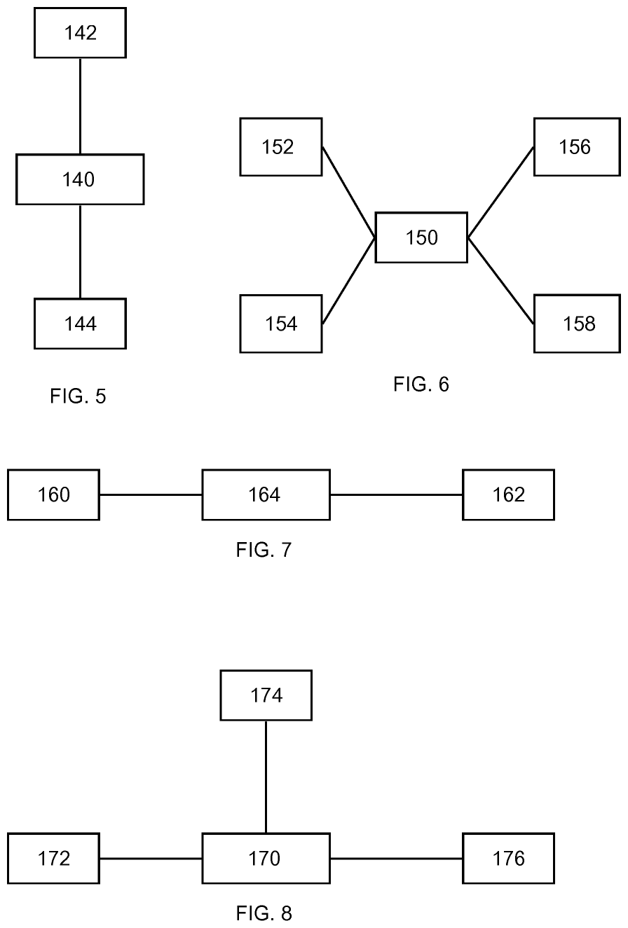

As illustrated in FIG. 5, the approximate propagation step (140) provides a computationally practical means of evolving the state over discrete intervals. The exponential operator e\hat{ }(−iH\hat{ }DNAΔt/h−bar) (142) is applied to advance the state, yielding an updated quantum state ψ(t+Δt) (144). This formulation enables iterative simulation of DNA dynamics across biologically relevant timescales.

As illustrated in FIG. 6, the time-dependent DNA Recognition Transform FDNA(E(t)) (150) maps evolving energy levels to functional outputs. The transform depends on the stabilization factor Sstab(t) (152), time-varying energy level En(t) (154), coherence constant Ecoh (156), and periodicity parameter P0 (158). This mapping yields time-dependent amplitude outputs |FDNA(E(t))|\hat{ }2 that directly correspond to observable biological effects such as transcription rates, coherence signatures, or regulatory activity.

The constructs LDNA(t), H\hat{ }DNA(t), and FDNA(E(t)) collectively extend DNARP into a dynamic framework. Sequence accessibility S(t) may change due to binding or modification events, shape H(t) may change due to conformational adjustments, and energy E(t) may change through excitation or relaxation processes. These dynamic constructs enable the invention to capture time-dependent biological phenomena using Recognition Physics principles without reliance on empirical parameter fitting.

The invention further provides a method for simulating the dynamics of biological processes using the RS-based DNARP framework. The method is executed computationally and applies the time-dependent constructs described above to iteratively predict biological behavior.

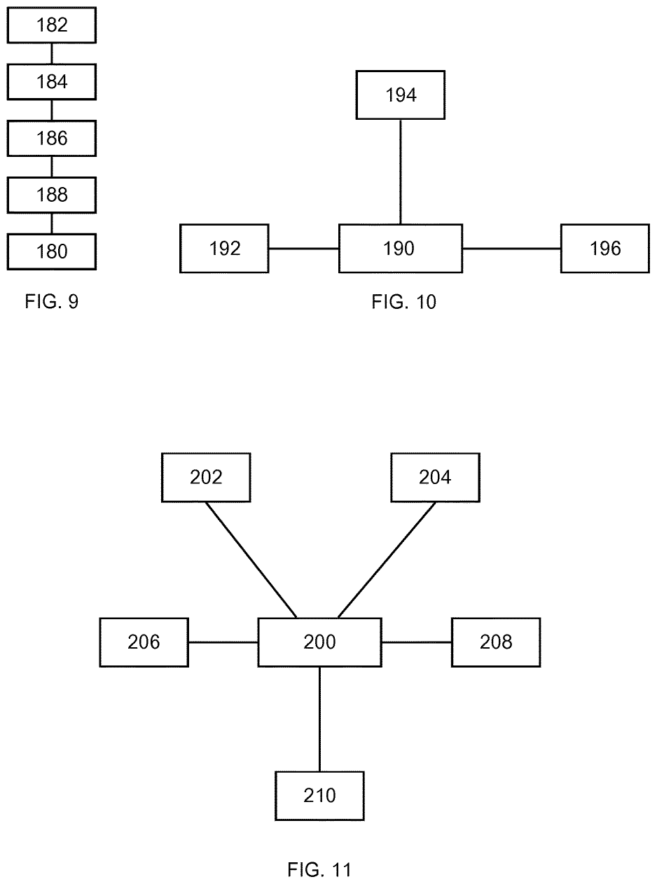

As illustrated in FIG. 9, the method flowchart (180) begins with initialization of the DNARP state (182). In this step, an initial triplet D(t0)=(S(t0), H(t0), E(t0)) is defined based on recognition physics parameters and validated for stability using the static constructs LDNA, H\hat{ }DNA, and FDNA.

The next step involves definition of RS-based evolution rules (184). These rules specify how one or more components of the triplet D(t) will change in response to biological interactions or stimuli. Examples include modification of sequence accessibility S(t) due to repressor binding, conformational change of shape H(t) due to supercoiling, or transition of energy state E(t) due to excitation or relaxation events.

The dynamic simulation then proceeds through a time evolution loop (186). At each time step, the evolution rules are applied to update S(t), H(t), and E(t). The updated values are then substituted into the governing constructs LDNA(t), H\hat{ }DNA(t), and FDNA(E(t)). Where required, the Schrödinger-like equation of FIG. 4 is solved or the propagation step of FIG. 5 is applied to evolve the state ψ(x, t). The result is a continuous or discrete progression of the DNA state over time, reflecting the defined biological context.

The final step of the method is output prediction (188). At each iteration of the time evolution, the functional output is calculated using FDNA(E(t)), producing time-series data such as gene expression rate, coherence intensity, or protein concentration. These predicted outputs provide a parameter-free forecast of biological dynamics.

By integrating initialization, rule definition, time evolution, and output prediction, the method enables computational simulation of DNA dynamics and related biological processes. Importantly, the method does not rely on empirical fitting of kinetic parameters but instead derives from RS-based governing constructs, ensuring reproducibility and predictive accuracy across contexts.

In another aspect, the invention provides a computational system configured to implement the method described above. The system is designed to manage input definitions, perform time-dependent simulation, and generate outputs reflecting predicted biological dynamics.

As illustrated in FIG. 8, the system architecture diagram (170) comprises an input module (172), a dynamic simulation engine (174), and an output module (176). These modules are implemented on a processor and memory architecture capable of storing, evolving, and reporting the DNARP state.

The input module (172) enables a user or external software to define the initial DNARP state D(t0)=(S(t0), H(t0), E(t0)) and specify RS-based evolution rules governing its progression. The input module may accept definitions in textual, graphical, or symbolic form, and validates the initial state against static RS constructs to ensure internal consistency.

The dynamic simulation engine (174) forms the core of the system. It applies the defined evolution rules over successive time steps, updating the components of the DNARP state. Within the engine, submodules are responsible for solving the time-dependent DNA Lagrangian LDNA(t), applying the DNA operator H\hat{ }DNA(t), and calculating the DNA Recognition Transform FDNA(E(t)). The engine may implement either exact solutions or approximate propagation steps, such as the exponential operator described in FIG. 5, depending on computational requirements.

The output module (176) stores and presents the predicted results of the simulation. This may include time-series data of sequence accessibility S(t), conformation H(t), energy coherence levels E(t), and associated functional outputs such as transcription rate, coherence amplitude, or regulatory activity. The output module may provide results in graphical form, exportable data files, or integration with external biological modeling platforms.

The system architecture provides a complete framework for parameter-free biological simulation. By coupling input definition, iterative RS-based evolution, and output reporting, the system enables practical deployment of the invention for research, clinical, or industrial applications.

The invention further provides a schematic representation of DNA dynamics, illustrating how the sequence, shape, and energy components evolve under time-dependent conditions. This schematic captures the integrated behavior of the DNARP triplet as it responds to biological interactions or environmental inputs.

As illustrated in FIG. 10, the DNA dynamics schematic (190) depicts the evolution of sequence accessibility S(t) (192), DNA shape H(t) (194), and coherence energy E(t) (196). These three components are represented as interdependent variables whose values change during the course of simulation.

The sequence component S(t) (192) reflects the accessibility of nucleotide regions. Binding of repressors or activators, or modifications such as methylation, can alter effective accessibility without altering the underlying base sequence. The schematic illustrates how such recognition events modify S(t) during simulation.

The shape component H(t) (194) reflects the conformational state of the DNA molecule. Changes such as supercoiling, groove width variation, or protein-induced bending shift the periodicity and geometric ratios of DNA structure. These conformational changes are incorporated into the simulation as modifications of H(t), as illustrated in the schematic.

The energy component E(t) (196) reflects the coherence state of the DNA system. Excitation by photons, binding events, or stress signals can increase the coherence level, while relaxation processes decrease it. The schematic shows transitions between energy states, which correspond to quantized coherence levels defined by the RS operator constructs.

Together, sequence accessibility S(t) (192), DNA shape H(t) (194), and coherence energy E(t) (196) provide a unified representation of DNA dynamics within the RS-based simulation framework. FIG. 10 illustrates how changes in any one component can propagate to the others, thereby capturing the coupled nature of biological processes.

To illustrate the application of the invention, an example simulation of gene activation is provided. This example demonstrates how the RS-based DNARP framework models the effect of an activator molecule on DNA dynamics and predicts resulting changes in expression rate.

As illustrated in FIG. 11, the example application (200) begins with an activator molecule (202) binding to a DNA region (204). The binding event is defined as an RS-based evolution rule that modifies the potential term within the DNA operator H\hat{ }DNA(t).

Prior to activation, the DNA coherence state is represented as E1 (206), corresponding to a baseline energy of approximately 0.09 electron volts and an expression rate of 50 bases per second. When the activator molecule (202) binds, the rule specifies an induced transition to the higher coherence state E2 (208). This transition doubles the coherence energy to approximately 0.18 electron volts, increasing the rate of base incorporation to 100 bases per second.

The Recognition Transform FDNA(E(t)) maps the updated energy level to an amplitude output, producing an intensity proportional to |FDNA(E2)|². In this example, the calculated value corresponds to a rate increase to approximately 225 bases per second, exceeding the baseline transcription rate by more than fourfold.

A feedback rule may also be applied, such that when the predicted output rate exceeds a threshold of 200 bases per second, the stabilization factor Sstab is increased by 0.5 units. This feedback modifies the system state, influencing subsequent rounds of simulation and demonstrating how the invention captures regulatory loops within biological processes.

The gene activation schematic of FIG. 11 thus illustrates the ability of the invention to model recognition-driven transitions in DNA coherence states and predict their impact on functional outputs. The example confirms that the system provides parameter-free prediction of time-dependent biological dynamics in response to molecular interactions.

In one embodiment, the invention employs a set of RS-derived constants that are defined without reliance on empirical parameter fitting. These constants provide the foundation for all DNARP constructs and ensure parameter-free reproducibility of the invention.

The coherence constant Ecoh (126, 156) is approximately 0.090 electron volts, derived from the golden ratio phi raised to the negative fifth power. Ecoh defines the discrete energy spacing between coherence levels of the DNA system.

The fundamental tick constant tau₀ is approximately 7.33×10⁻¹⁵ seconds when mapped to SI units. Tau₀ represents the minimal recognition interval that bounds temporal evolution within the RS framework.

The golden ratio phi is approximately 1.618, serving as a fixed point constant across RS derivations. Phi appears in scaling laws and defines self-similar relationships in the spectra of DNA dynamics.

The DNA length constant XDNA (116) is approximately 13.6 Ångstroms, representing a characteristic spatial scale derived from RS recognition principles.

The periodicity constant P₀ (128, 158) is approximately 35.6 Ångstroms, representing a fundamental DNA groove periodicity derived from RS constructs.

By incorporating Ecoh (126, 156), tau₀, phi, XDNA (116), and P₀ (128, 158) into the DNARP framework, the invention ensures that all simulations are grounded in RS-derived constants. These constants provide the quantitative foundation for stability, coherence, and recognition transforms, and distinguish the invention from conventional models that rely on empirical parameter fitting.

In another embodiment, the invention is applied to the simulation of circadian rhythm dynamics. Circadian rhythms are biological oscillations with an approximately twenty-four-hour period that regulate gene expression, metabolic activity, and cellular behavior. Modeling such processes requires capturing both periodicity and feedback-driven transitions in DNA dynamics.

As applied in the present invention, the DNARP state D(t)=(S(t), H(t), E(t)) is initialized to represent a baseline genetic program associated with circadian regulation. Evolution rules are defined to model oscillatory protein binding events that periodically modify sequence accessibility S(t) (192), shape conformation H(t) (194), or coherence energy E(t) (196).

The time evolution of the DNARP state is computed using the governing constructs LDNA(t), H\hat{ }DNA(t), and FDNA(E(t)). Oscillatory changes in S(t), H(t), and E(t) produce predicted time-series outputs that exhibit periodic fluctuations in gene expression intensity. The invention thus simulates circadian rhythm dynamics as recurring transitions between coherence states.

By applying parameter-free RS constructs, the invention provides a first-principles model of circadian oscillations that does not rely on empirical curve-fitting of rate constants. The results capture the phase, amplitude, and period of circadian gene regulation, demonstrating that the framework applies not only to single interaction events such as gene activation but also to longer-term oscillatory biological processes.

In further embodiments, additional systems, methods, architectures, processing pipelines, and implementation arrangements are provided for simulating and predicting biological process dynamics using the Recognition Physics (RS)-based DNA Recognition Physics framework described herein. These further embodiments may extend, refine, or elaborate upon the previously described formulations, figures, and computational arrangements, while remaining consistent with the dynamic DNARP representation in which a biological process state is represented as D(t)=(S(t), H(t), E(t)). In this representation, S(t) may correspond to a time-dependent sequence accessibility component, H(t) may correspond to a time-dependent structural or conformational component, and E(t) may correspond to a time-dependent energy or coherence component.

In some embodiments, the further implementations described below may be used in conjunction with one or more of the previously described static and dynamic governing constructs. For example, the time-dependent Schrödinger-like equation (130), the time-dependent DNA operator (136), the approximate propagation step (140), and the time-dependent DNA Recognition Transform (150) may be used individually or in combination to evolve a time-dependent biological state and to generate one or more predicted outputs over time. In some embodiments, the dynamic state D(t) may be initialized from an initial state D(t0), and may thereafter be iteratively updated across one or more time intervals according to one or more Recognition Physics (RS)-based evolution rules, transition conditions, constraints, or operational settings.

In some embodiments, the further implementations may provide additional detail regarding how the dynamic DNARP state is received, validated, updated, propagated, and reported in a computational setting. Thus, while previously described embodiments set forth the governing framework, the present further embodiments may describe more detailed subsystem arrangements, rule-processing arrangements, solver arrangements, time-stepping arrangements, and output-generation arrangements for carrying out simulations of biological process dynamics. Such embodiments may be implemented as refinements of, extensions of, or alternative implementations of the previously described computational system architecture (170), including the input module (172), the dynamic simulation engine (174), and the output module (176).

In some embodiments, the further implementations may also provide additional detail regarding how Recognition Physics (RS)-based evolution rules are defined and applied to the dynamic DNARP state. For example, such rules may specify how one or more components of D(t) change in response to internal biological conditions, external perturbations, binding events, conformational adjustments, coherence transitions, regulatory interactions, environmental influences, epigenetic changes, drug exposure, repair activity, oscillatory drivers, or combinations thereof. In some embodiments, one or more such rules may affect sequence accessibility S(t) (192), DNA shape H(t) (194), and coherence energy E(t) (196), either independently or in coupled fashion, over one or more simulation intervals.

In some embodiments, the further implementations may provide additional detail regarding how the updated values of S(t), H(t), and E(t) are supplied to one or more governing constructs to produce dynamic predictions. For example, updated state values may be substituted into the time-dependent constructs and used to compute evolved state information, transformed output information, intermediate state histories, or other simulation data products. In some embodiments, such outputs may include one or more predicted output rates, trajectories, time-series values, transition histories, stability indicators, coherence-state progressions, or other representations of simulated biological behavior. These outputs may be generated at each iteration of a simulation, at selected intervals, upon occurrence of triggering events, or upon satisfaction of one or more reporting conditions.

In some embodiments, the further implementations may be used to simulate biological dynamics across a variety of contexts. By way of example and not limitation, the disclosed framework may be used for simulating or predicting gene regulation dynamics, drug response behavior, environmental stress response, circadian behavior, DNA repair activity, chromatin or epigenetic response, regulatory-state transitions, or other biological processes involving time-dependent changes in DNA-associated state variables. In some embodiments, these differing contexts may be represented through different initial states, different evolution rules, different boundary conditions, different solver selections, different reporting outputs, or combinations thereof, while still using the same underlying Recognition Physics (RS)-based dynamic framework.

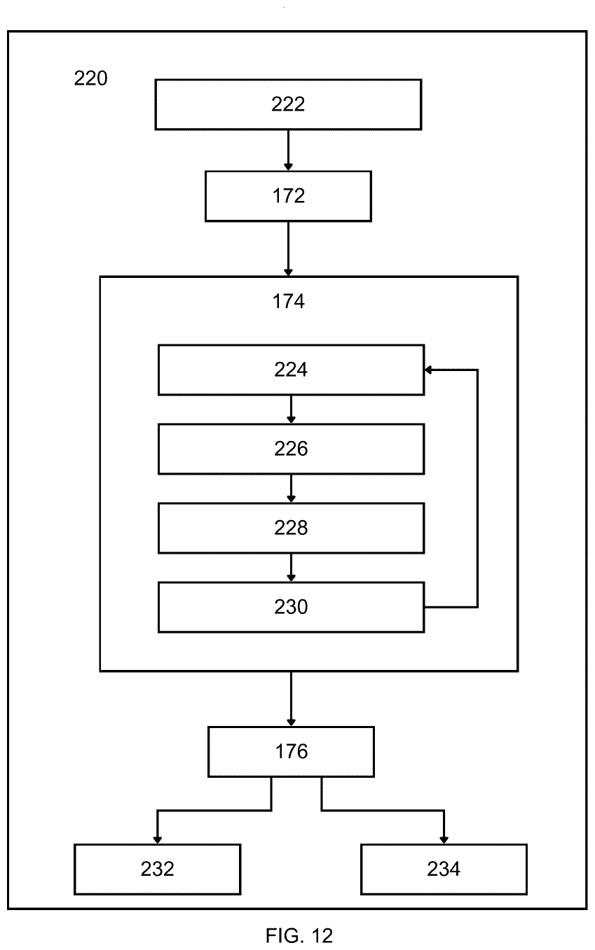

In some embodiments, the further implementations may also provide additional internal architectural detail for carrying out the dynamic simulations disclosed herein. For example, and as described in greater detail below with reference to FIG. 12, an expanded computational system architecture (220) may receive an initial DNARP state and evolution rule input (222), and may process such input through one or more internal components including a state manager (224), a rule engine (226), one or more RS construct solvers (228), and a time stepper (230), operating within or as part of the dynamic simulation engine (174). In some embodiments, these internal components may cooperate to maintain a current simulation state, evaluate and apply applicable evolution rules, update one or more governing constructs, propagate the biological state across time, and provide output information to the output module (176) for generation of predicted output time-series (232) and state-variable output data (234).

In some embodiments, the further implementations may also provide greater detail regarding the operational relationship between the previously described method flowchart (180) and the expanded computational arrangements described herein. For example, the initialization step (182), the definition of evolution rules (184), the time evolution loop (186), and the output prediction step (188) may each be carried out through more detailed internal processing stages, including validation of the initial state, parsing or evaluation of rule expressions, repeated updating of state variables, solver-based propagation of the time-dependent state, and storage or reporting of generated outputs. In this manner, the further embodiments may provide a more detailed implementation-level view of the same general dynamic simulation framework already introduced above.

In some embodiments, any one or more features described in the further embodiments may be used alone or in combination with one another, and may be combined with one or more features described elsewhere herein. Accordingly, the further embodiments should not be understood as limiting the scope of the disclosed system or method to any single architecture, rule format, solver arrangement, deployment configuration, biological context, or output format, unless expressly stated otherwise.

In some embodiments, and with reference to FIG. 12, an expanded computational system architecture (220) is provided as a more detailed subsystem-level implementation of the computational arrangements previously described with reference to FIG. 8. In this regard, the expanded computational system architecture (220) may be understood as a drill-down view of the previously described system architecture (170), and may preserve the same general top-level arrangement including an input module (172), a dynamic simulation engine (174), and an output module (176), while further specifying internal subsystem components and their functional relationships. In some embodiments, the expanded computational system architecture (220) may be used to implement the same Recognition Physics (RS)-based dynamic DNARP simulation framework previously described herein, but with added architectural detail regarding how the evolving state is managed, how evolution rules are evaluated, how the governing constructs are applied, and how simulation time is advanced.

In some embodiments, the expanded computational system architecture (220) may receive an initial DNARP state and evolution rule input (222). The initial DNARP state and evolution rule input (222) may include an initial state D(t0), one or more state-variable definitions, one or more simulation interval definitions, one or more rule definitions, one or more threshold conditions, one or more triggering events, one or more boundary conditions, one or more operational constraints, one or more output settings, or combinations thereof. In some embodiments, the initial state D(t0) may include a sequence accessibility component, a structural or conformational component, and an energy or coherence component, and may therefore define an initial DNARP triplet for simulation. In some embodiments, the evolution rule input may specify how one or more components of the DNARP state change over time in response to biological interactions, external perturbations, regulatory influences, periodic conditions, repair events, epigenetic conditions, or other recognized events or conditions.

In some embodiments, the input module (172) may receive, validate, normalize, parse, transform, or otherwise prepare the initial DNARP state and evolution rule input (222) for downstream processing. For example, the input module (172) may receive input in textual, symbolic, structured, graphical, machine-readable, or user-defined form, and may convert such input into one or more internal representations suitable for use by the dynamic simulation engine (174). In some embodiments, the input module (172) may validate the received initial state against one or more static or dynamic Recognition Physics (RS)-based constraints, may verify the internal consistency of one or more rule expressions, and may identify or reject incomplete, inconsistent, or incompatible inputs before simulation proceeds. In some embodiments, the input module (172) may further prepare scheduling information, event definitions, conditional expressions, solver settings, reporting settings, or other execution parameters for use by downstream subsystem components.

In some embodiments, the dynamic simulation engine (174) may comprise one or more internal subsystem components including a state manager (224), a rule engine (226), one or more RS construct solvers (228), and a time stepper (230). These subsystem components may operate in a serial arrangement, an iterative arrangement, a partially parallel arrangement, a recursive arrangement, or any coordinated combination thereof. In some embodiments, the dynamic simulation engine (174) may therefore serve as the core processing environment in which the evolving DNARP state is stored, updated, propagated, and converted into one or more predicted outputs.

In some embodiments, the state manager (224) may maintain, access, update, store, retrieve, or otherwise manage one or more current, prior, intermediate, and future state representations associated with the simulation. In some embodiments, the state manager (224) may maintain the current DNARP state D(t), including one or more values associated with sequence accessibility, structural or conformational shape, and energy or coherence condition. In some embodiments, the state manager (224) may also maintain one or more derived state values, transformed state values, historical state values, comparison states, candidate future states, or checkpoint states. By way of example, the state manager (224) may store one or more time-indexed values associated with stabilization-related quantities, periodicity-related quantities, coherence-state quantities, output-associated quantities, or combinations thereof. In some embodiments, the state manager (224) may provide the then-current state to the rule engine (226) for rule evaluation, may provide updated state values to the RS construct solvers (228) for construct computation, and may receive an updated propagated state for storage and use in a subsequent interval.

In some embodiments, the rule engine (226) may evaluate, select, apply, schedule, prioritize, or otherwise manage one or more Recognition Physics (RS)-based evolution rules associated with the simulation. In some embodiments, the rule engine (226) may determine whether one or more conditions for rule application are satisfied at a given simulation interval, event boundary, threshold crossing, or other processing point. In some embodiments, the rule engine (226) may apply one or more resulting actions to the then-current state, including actions affecting sequence accessibility, conformational geometry, structural periodicity, coherence transitions, interaction potentials, output-associated quantities, or combinations thereof. In some embodiments, the rule engine (226) may operate on the basis of deterministic logic, event-triggered logic, threshold-triggered logic, scheduled logic, probabilistic logic, or hybrid logic. In some embodiments, the rule engine (226) may therefore define how biological interactions or modeled perturbations are translated into simulation-state updates within the dynamic simulation engine (174).

In some embodiments, the one or more RS construct solvers (228) may compute, apply, solve, approximate, evaluate, or otherwise implement one or more governing constructs used in the time-dependent simulation. For example, the RS construct solvers (228) may evaluate one or more forms of the time-dependent Schrödinger-like equation (130), may apply the time-dependent DNA operator (136), may perform one or more approximate propagation operations associated with the approximate propagation step (140), may apply one or more exponential operator forms such as the exponential operator (142), and may determine one or more evolved state quantities corresponding to an evolved state (144). In some embodiments, the RS construct solvers (228) may further evaluate the time-dependent DNA Recognition Transform (150), including one or more values associated with the stabilization factor Sstab(t) (152), the energy level En(t) (154), the coherence constant Ecoh (156), the periodicity parameter P0 (158), or combinations thereof. In some embodiments, the RS construct solvers (228) may operate using exact solutions, approximate solutions, discretized solutions, propagator-based solutions, iterative solutions, operator-based solutions, or hybrid solution techniques, depending on implementation preference and computational requirements.

In some embodiments, the RS construct solvers (228) may receive updated state information from the state manager (224), receive rule-effect information from the rule engine (226), and compute one or more updated state values, transformed quantities, intermediate trajectories, output-associated values, or propagated state representations based on the governing constructs. In some embodiments, the RS construct solvers (228) may thereby provide a computational bridge between the current DNARP state, the rule-driven updates applied to that state, and the resulting propagated biological behavior predicted by the simulation. In some embodiments, one or more values generated by the RS construct solvers (228) may be returned to the state manager (224), passed to the output module (176), or supplied to the time stepper (230) for subsequent interval control.

In some embodiments, the time stepper (230) may control progression of the simulation across time. For example, the time stepper (230) may define, select, increment, adjust, or otherwise manage one or more simulation intervals, including fixed time steps, variable time steps, event-driven steps, adaptive steps, checkpoint intervals, or combinations thereof. In some embodiments, the time stepper (230) may coordinate the order in which rule evaluation, state update, construct computation, propagation, output capture, and loop continuation occur. In some embodiments, the time stepper (230) may determine whether a simulation continues, pauses, branches, terminates, or advances to a next interval based on one or more stop conditions, reporting conditions, event conditions, or control settings. In some embodiments, the time stepper (230) may also coordinate the return of an updated state representation to the state manager (224) for use in a subsequent interval, thereby supporting iterative, loop-based state evolution over time.

In some embodiments, the output module (176) may receive one or more outputs from one or more internal components of the dynamic simulation engine (174), including the state manager (224), the rule engine (226), the RS construct solvers (228), and the time stepper (230). In some embodiments, the output module (176) may generate, store, transmit, render, export, visualize, summarize, or otherwise provide one or more outputs including predicted output time-series (232) and state-variable output data (234). In some embodiments, the predicted output time-series (232) may include one or more predicted functional outputs represented across time, such as one or more rates, amplitudes, concentrations, activation levels, coherence-related outputs, regulatory outputs, or other time-dependent biological outputs. In some embodiments, the state-variable output data (234) may include one or more time-indexed or event-indexed values associated with the evolving DNARP state, including one or more values corresponding to sequence accessibility, structural or conformational state, energy or coherence state, derived transform values, or other simulation-associated variables.

In some embodiments, the output module (176) may provide output information in graphical form, numerical form, tabular form, file-based form, machine-readable form, dashboard form, report form, or forms suitable for downstream integration with one or more external systems. In some embodiments, the output module (176) may support generation of time-series visualizations, state trajectories, summary metrics, comparative results, alerts, flags, exported data packages, or other reporting artifacts useful for research, modeling, validation, intervention design, or downstream computational analysis. In some embodiments, the output module (176) may further support storage of intermediate values, final results, and simulation histories for later retrieval, reprocessing, comparison, or audit.

In some embodiments, the subsystem components shown in FIG. 12 may be physically separated, logically separated, partially integrated, fully integrated, distributed across multiple computational resources, virtualized, containerized, replicated, or otherwise arranged according to implementation preference. Accordingly, although the expanded computational system architecture (220) is shown as including the state manager (224), the rule engine (226), the RS construct solvers (228), and the time stepper (230) within the dynamic simulation engine (174), other arrangements may also be used. For example, one or more of the depicted subsystem components may be combined into a shared processing module, subdivided into additional submodules, or implemented as callable services, libraries, routines, engines, or execution layers.

In some embodiments, the expanded computational system architecture (220) may therefore provide a more detailed internal architectural view of the computational system previously introduced with reference to FIG. 8, while remaining consistent with the same underlying Recognition Physics (RS)-based dynamic DNARP framework. In this manner, FIG. 12 may be understood as showing one exemplary subsystem-level implementation in which initial state and rule information are received through the input module (172), processed through the dynamic simulation engine (174) and its internal subsystem components, and provided to the output module (176) for generation of predicted output time-series (232) and state-variable output data (234).

In some embodiments, the dynamic simulation framework described herein may be implemented through a Recognition Physics (RS)-based processing pipeline that operates over time to receive an initial biological state, apply one or more evolution rules, update one or more governing constructs, propagate the state across one or more simulation intervals, and generate one or more predicted outputs. With reference to FIG. 9, such a pipeline may correspond generally to the previously described method flowchart (180), including initialization (182), definition of evolution rules (184), execution of a time evolution loop (186), and output prediction (188). In some embodiments, and with further reference to FIG. 12, the same general method may be carried out through the coordinated operation of the expanded computational system architecture (220), including the input module (172), the dynamic simulation engine (174), the state manager (224), the rule engine (226), the RS construct solvers (228), the time stepper (230), and the output module (176).

In some embodiments, the processing pipeline may begin with an initialization phase in which an initial DNARP state is received and established for simulation. For example, the initialization step (182) may define an initial state D(t0) corresponding to a biological process at an initial time. In some embodiments, D(t0) may include one or more initial values associated with sequence accessibility, structural or conformational state, and energy or coherence state, such that the simulation begins with an initial DNARP triplet. In some embodiments, the initial DNARP state and evolution rule input (222) received by the expanded computational system architecture (220) may further include one or more simulation interval settings, one or more initial conditions, one or more triggering conditions, one or more solver settings, one or more stopping conditions, one or more reporting preferences, or combinations thereof. In some embodiments, the input module (172) may receive, normalize, validate, parse, or otherwise prepare this initial information for downstream execution, and the state manager (224) may store the validated initial state as the then-current state to be used during subsequent simulation intervals.

In some embodiments, following initialization, the processing pipeline may proceed to a rule-definition or rule-ingestion phase. With reference to FIG. 9, the definition of evolution rules (184) may include establishing one or more Recognition Physics (RS)-based rules that govern how the dynamic state changes over time. In some embodiments, such rules may be expressed in conditional form, event-driven form, threshold-based form, time-based form, schedule-based form, probabilistic form, deterministic form, or combinations thereof. In some embodiments, the rule engine (226) may receive, parse, transform, prioritize, or otherwise manage such rule expressions as part of the execution pipeline. By way of example, one or more rules may specify changes to sequence accessibility resulting from binding events, modification events, or regulatory-state changes, may specify changes to structural or conformational state resulting from supercoiling, local bending, compaction, protein-induced deformation, or other structural influences, and may specify changes to energy or coherence state resulting from excitation, relaxation, interaction, decoherence, or other energetic or coherence-related events. In some embodiments, the rule engine (226) may determine how such rule definitions are represented internally and how such rules are evaluated against the then-current state at each relevant simulation interval.

In some embodiments, once the initial state and applicable rules have been established, the processing pipeline may enter the time evolution loop (186). In some embodiments, the time evolution loop (186) may include repeatedly evaluating the then-current state, determining whether one or more rules are active, applying one or more state-changing actions associated with active rules, updating one or more inputs to the governing constructs, propagating the state to a subsequent interval, and determining whether the simulation is to continue. In some embodiments, the state manager (224), the rule engine (226), the RS construct solvers (228), and the time stepper (230) may cooperatively perform these operations in an iterative manner. For example, the state manager (224) may provide the then-current state to the rule engine (226), the rule engine (226) may determine which rule conditions are satisfied and what updates are to be applied, the RS construct solvers (228) may compute one or more propagated state quantities based on the updated inputs, and the time stepper (230) may advance the simulation to a subsequent time interval and coordinate repetition of the loop.

In some embodiments, the time evolution loop (186) may include rule-triggered updates to one or more components of the DNARP state. For example, the rule engine (226) may determine that a particular event, threshold, or scheduled condition modifies one or more values associated with sequence accessibility, structural state, or energy state, and may communicate such modifications for further processing. In some embodiments, such updates may occur independently for individual state components or may occur in coupled fashion such that a change in one component causes or contributes to a change in one or more other components. In some embodiments, one or more rule applications may also depend on prior state history, elapsed time, repeated occurrence of an event, external input conditions, output-derived feedback, or combinations thereof.

In some embodiments, following one or more rule-driven updates, the processing pipeline may update and apply one or more governing constructs used to propagate the biological state. For example, updated state values may be substituted into one or more time-dependent constructs including the time-dependent Schrödinger-like equation (130), the time-dependent DNA operator (136), and the time-dependent DNA Recognition Transform (150). In some embodiments, the RS construct solvers (228) may compute, approximate, or otherwise evaluate one or more propagation operations associated with the approximate propagation step (140), the exponential operator (142), and the evolved state (144). In some embodiments, the RS construct solvers (228) may further evaluate one or more transform-associated values including the stabilization factor Sstab(t) (152), the energy level En(t) (154), the coherence constant Ecoh (156), the periodicity parameter P0 (158), or related quantities. In some embodiments, the governing constructs may therefore serve as the mathematical or computational mechanism by which the updated DNARP state is propagated from one interval to the next and by which one or more predicted biological outputs are derived from the evolving state.

In some embodiments, the RS construct solvers (228) may recompute one or more structural values, operator values, transform values, propagated state values, derived quantities, or output-associated values at each time step, at selected time steps, upon occurrence of triggering events, upon change of one or more solver conditions, or according to other execution logic. In some embodiments, the propagated state produced by the RS construct solvers (228) may be returned to the state manager (224) for storage as the updated current state. In some embodiments, the time stepper (230) may determine whether the updated state is to be used immediately for a next iteration, whether a reporting operation is to be performed, whether one or more branch conditions are satisfied, or whether one or more stop conditions have been reached.

In some embodiments, the processing pipeline may further include output generation and reporting. With reference to FIG. 9, the output prediction step (188) may calculate or otherwise determine one or more functional outputs from the evolved biological state. In some embodiments, and with reference to FIG. 12, the output module (176) may receive one or more values, trajectories, or derived quantities from the dynamic simulation engine (174) and may generate predicted output time-series (232) and state-variable output data (234). In some embodiments, the predicted output time-series (232) may include one or more predicted biological, biochemical, regulatory, or coherence-related outputs across time, including by way of example one or more predicted expression rates, regulatory-state outputs, response amplitudes, interaction-associated outputs, or other time-dependent outputs. In some embodiments, the state-variable output data (234) may include one or more values or trajectories associated with the evolving DNARP state, such as time-indexed values corresponding to sequence accessibility, structural or conformational state, energy or coherence state, transformed quantities, derived metrics, or other simulation-associated variables.

In some embodiments, the output module (176) may generate outputs at each time interval, at selected intervals, upon satisfaction of one or more reporting conditions, upon occurrence of one or more triggering events, or upon completion of the simulation. In some embodiments, output information may be stored, transmitted, rendered, visualized, exported, summarized, or otherwise made available for downstream use. For example, the output module (176) may provide numerical outputs, graphical trajectories, tabular summaries, machine-readable files, comparative reports, dashboard views, alerts, annotations, or other reporting artifacts corresponding to the simulation results. In some embodiments, intermediate state histories, output histories, confidence indicators, validation flags, exception indicators, rule-activation histories, or derived summaries may also be stored or reported as part of the pipeline.

In some embodiments, the processing pipeline may further support iterative feedback behavior. For example, one or more outputs generated during one interval or one stage of execution may act as inputs, conditions, or triggers for subsequent rule evaluation during a later interval. In some embodiments, a generated state-variable output, a transformed quantity, or a predicted functional output may satisfy a threshold or condition that activates an additional rule, suppresses a previously active rule, modifies a time-stepping setting, changes a solver configuration, or otherwise alters subsequent execution of the pipeline. In this manner, the Recognition Physics (RS)-based processing pipeline may support not only forward propagation of an evolving biological state, but also feedback-aware iterative updating in which prior outputs influence later state transitions.

In some embodiments, the processing pipeline may continue until one or more completion conditions are satisfied. For example, the time stepper (230) may determine that a terminal time has been reached, that a convergence condition has been satisfied, that a triggering event has terminated the simulation, that an intervention point has been reached, that a reporting objective has been completed, or that another stop condition has occurred. Upon termination, final outputs may be generated and stored, and one or more intermediate histories may be preserved for later review, comparison, replay, audit, or reuse. Accordingly, the Recognition Physics (RS)-based processing pipeline described herein may provide an implementation-level framework through which the previously introduced dynamic DNARP methodology may be carried out in a structured, iterative, and computationally executable manner.

In some embodiments, the expanded computational system architecture (220) and the Recognition Physics (RS)-based processing pipeline described herein may be configured for different biological process modalities by varying one or more initial-state definitions, evolution rules, triggering conditions, boundary conditions, solver settings, reporting settings, output formats, or combinations thereof. In this manner, the same underlying Recognition Physics (RS)-based dynamic DNARP framework may be adapted for different categories of biological dynamics while preserving the general architectural and computational relationships described above. For example, different implementations may employ different initial values for sequence accessibility, structural or conformational state, and energy or coherence state, may define different rule sets for how such values evolve over time, and may generate different classes of predicted outputs depending on the biological process being simulated.

In some embodiments, the recognition subsystem architecture may be configured for gene regulation implementations. For example, and with reference to FIG. 11, a gene activation application (200) may include a DNA promoter region (202), a transcription factor interaction (204), a dynamic state evolution process (206), a time-varying gene expression rate (208), and a gene activation output (210). In some embodiments, the initial state for such an implementation may include one or more values associated with promoter accessibility, local DNA conformation, chromatin-related context, binding readiness, and one or more energy or coherence-related conditions associated with transcriptional activation. In some embodiments, the rule engine (226) may evaluate one or more rules associated with transcription-factor binding, transcription-factor release, repressor activity, enhancer-related influence, chromatin opening, chromatin closing, or other regulatory events. In some embodiments, such rules may modify one or more components of the dynamic state, including sequence accessibility, structural conformation, and coherence-related energy conditions, and the RS construct solvers (228) may propagate the resulting updated state across one or more simulation intervals. In some embodiments, the output module (176) may generate predicted output time-series (232) corresponding to one or more gene expression trajectories, activation-state transitions, repression-state intervals, timing relationships, or output-associated values, and may further generate state-variable output data (234) reflecting one or more changes in the evolving regulatory state. In this regard, the architecture may simulate not only whether a gene is activated or repressed, but also how the activation or repression behavior unfolds dynamically over time.

In some embodiments, the recognition subsystem architecture may be configured for drug response implementations. In some embodiments, a drug exposure may be represented as an external perturbation, binding event, environmental condition, or state-modifying influence that changes one or more components of the dynamic DNARP state. For example, a drug may alter sequence accessibility by affecting binding conditions, may alter DNA shape or related structural features by inducing compaction, bending, torsional change, or interaction with associated molecules, and may alter the energy or coherence component by introducing one or more stabilizing, destabilizing, activating, inhibitory, or transition-producing influences. In some embodiments, the initial state may include a pre-exposure biological state, and one or more evolution rules may define how the state changes upon drug introduction, continued exposure, dose variation, repeated administration, clearance, or interaction with another biological factor. In some embodiments, the rule engine (226) may apply one or more drug-related rules, the RS construct solvers (228) may compute corresponding propagated effects, and the output module (176) may generate predicted output time-series (232) representing one or more time-varying response profiles, dose-response trajectories, delayed-response behaviors, transient activation states, or recovery profiles. In some embodiments, state-variable output data (234) may indicate one or more drug-induced changes in accessibility, structure, coherence, transform-associated quantities, or related simulation variables over time.

In some embodiments, the recognition subsystem architecture may be configured for environmental stress implementations. For example, heat shock, oxidative stress, nutrient deprivation, pH variation, radiation exposure, inflammatory signaling, mechanical stress, or other environmental perturbations may act as external triggers that alter one or more components of the dynamic state. With reference to FIG. 10, such implementations may be described in relation to a time-dependent DNARP representation (190), including sequence accessibility (192), DNA shape (194), and coherence energy (196). In some embodiments, the initial state may represent a baseline biological condition prior to a stress event, and one or more evolution rules may define how accessibility, conformation, and energy-related state values change in response to the onset, duration, severity, periodicity, or cessation of a stress condition. In some embodiments, the rule engine (226) may determine that one or more stress-trigger conditions are satisfied and may apply corresponding state updates, while the RS construct solvers (228) may compute the propagated consequences of such updates and the time stepper (230) may coordinate stress-response evolution over one or more intervals. In some embodiments, the output module (176) may generate predicted output time-series (232) corresponding to one or more stress-response behaviors, adaptation trajectories, resilience measures, transition intervals, or failure-related outputs, and may further generate state-variable output data (234) reflecting changes in accessibility, conformational state, energy-related state, or related simulation quantities.

In some embodiments, the recognition subsystem architecture may be configured for circadian rhythm implementations. In some embodiments, the biological process being simulated may be influenced by one or more periodic, oscillatory, or scheduled conditions, and the associated evolution rules may therefore be defined in repeating, phase-dependent, time-window-dependent, or cycle-dependent form. For example, one or more rules may be scheduled to activate or deactivate at recurring intervals, may vary in strength as a function of phase position, or may interact with one or more external timing cues. In some embodiments, the initial state may represent a biological condition at a selected circadian phase, and the time stepper (230) may be configured to manage long-horizon or periodic simulation intervals suitable for modeling repeated cycles. In some embodiments, the rule engine (226) may apply one or more oscillatory rule sets that affect accessibility, conformation, and energy or coherence conditions over repeated intervals, and the RS construct solvers (228) may propagate the resulting state across one or more full or partial circadian cycles. In some embodiments, the output module (176) may generate predicted output time-series (232) showing one or more oscillatory outputs, periodic activation windows, phase-shifted responses, entrainment-related changes, amplitude variations, or other cycle-dependent biological behaviors, together with state-variable output data (234) showing how the underlying dynamic state evolves over time.

In some embodiments, the recognition subsystem architecture may be configured for DNA repair implementations. In some embodiments, a repair process may be represented as a sequence of state transitions associated with a damage event, a recognition event, an initiation event, one or more intermediate repair events, and a restoration or post-repair state. In some embodiments, the initial state may represent an undamaged baseline state or a damaged state existing at a selected initial time, and one or more evolution rules may define how the dynamic state changes upon damage detection, recruitment of repair-associated factors, local conformational change, altered accessibility, energetic transition, or restoration of a repaired condition. In some embodiments, the state manager (224) may track one or more pre-damage, damaged, intermediate repair, and repaired states across time, while the rule engine (226) may evaluate the conditions under which the simulation moves from one repair stage to another. In some embodiments, the RS construct solvers (228) may compute propagated state transitions associated with repair progression, and the output module (176) may generate predicted output time-series (232) corresponding to one or more repair-completion trajectories, repair-efficiency measures, delay intervals, pathway-choice outputs, or restoration-related outcomes. In some embodiments, state-variable output data (234) may indicate one or more accessibility changes, conformation changes, coherence changes, transform-related values, or other state-associated measures linked to the repair process.

In some embodiments, the recognition subsystem architecture may be configured for epigenetic or chromatin-related implementations. In some embodiments, methylation, acetylation, phosphorylation, chromatin compaction, chromatin relaxation, nucleosome repositioning, accessibility shifts, or related epigenetic or chromatin-associated events may be represented as one or more rules acting on the dynamic DNARP state. For example, one or more such events may affect sequence accessibility directly, may alter structural or conformational state by changing local compaction or geometry, and may affect energy or coherence conditions by altering one or more state transition relationships. In some embodiments, the initial state may include one or more epigenetic marks, chromatin-state indicators, accessibility values, structural values, or coherence-related values associated with a particular genomic region or biological context. In some embodiments, the rule engine (226) may evaluate one or more mark-dependent or chromatin-dependent rules, the RS construct solvers (228) may compute the resulting propagated state transitions, and the output module (176) may generate predicted output time-series (232) corresponding to one or more accessibility trajectories, chromatin-state changes, regulatory outcomes, persistence intervals, or reconfiguration behaviors. In some embodiments, state-variable output data (234) may indicate one or more changes in accessibility, structural state, energy or coherence condition, or related transform-associated values over time.

In some embodiments, two or more of the foregoing modality-specific implementations may be combined within a single simulation. For example, a gene regulation implementation may also include one or more drug response rules, one or more environmental stress triggers, one or more circadian timing conditions, one or more repair-related transitions, or one or more epigenetic-state modifications. In some embodiments, the initial state may therefore encode multiple overlapping biological conditions, and the rule engine (226) may evaluate multiple interacting rule sets during the same simulation. In some embodiments, the RS construct solvers (228) may propagate a state that reflects coupled influences across modalities, and the output module (176) may generate predicted output time-series (232) and state-variable output data (234) reflecting the combined or interacting effects of such conditions. Accordingly, the modality-specific implementations described herein should be understood as exemplary and non-limiting, and as demonstrating how the same recognition subsystem architecture may be adapted across a wide variety of biological process contexts while preserving the common Recognition Physics (RS)-based dynamic DNARP framework.

In some embodiments, the systems, methods, architectures, and processing pipelines described herein may be implemented using one or more computing devices, processors, memories, storage resources, interfaces, and execution environments configured to carry out the disclosed Recognition Physics (RS)-based dynamic DNARP simulations. In some embodiments, one or more functions of the input module (172), the dynamic simulation engine (174), the output module (176), the expanded computational system architecture (220), the state manager (224), the rule engine (226), the RS construct solvers (228), and the time stepper (230) may be implemented by one or more processors executing instructions stored in one or more memories or other storage media. In some embodiments, the disclosed functions may additionally or alternatively be implemented in hardware, firmware, configurable logic, dedicated circuitry, programmable logic devices, accelerator resources, or combinations thereof.

In some embodiments, one or more processors may execute instructions for receiving an initial DNARP state and evolution rule input, maintaining one or more time-indexed state representations, evaluating one or more Recognition Physics (RS)-based evolution rules, applying one or more governing constructs, propagating one or more biological states across time, generating one or more predicted outputs, storing one or more intermediate or final results, and presenting or exporting one or more output data products. In some embodiments, the memory resources used in connection with such processing may include volatile memory, non-volatile memory, cache memory, buffer memory, shared memory, distributed memory, persistent storage, or combinations thereof. In some embodiments, such memory resources may store executable instructions, simulation settings, state histories, rule definitions, solver configurations, transformed quantities, output histories, metadata, audit data, or other information associated with operation of the disclosed architecture.

In some embodiments, the input module (172), the dynamic simulation engine (174), the output module (176), the state manager (224), the rule engine (226), the RS construct solvers (228), and the time stepper (230) may be implemented as separate software modules, separate hardware modules, integrated modules, shared services, callable routines, execution layers, containers, threads, processes, libraries, engines, or combinations thereof. In some embodiments, one or more of such components may be combined into a common executable environment, and in other embodiments one or more such components may be separated across multiple computational resources. For example, the rule engine (226) and the RS construct solvers (228) may be integrated into a common processing engine in some implementations, while in other implementations the state manager (224), the rule engine (226), and the time stepper (230) may be deployed as separate services that communicate through one or more internal interfaces or message-passing mechanisms. Accordingly, the particular partitioning of the disclosed functionality into modules should be understood as exemplary rather than limiting.

In some embodiments, the disclosed systems may be implemented in a local execution environment, such as a workstation, desktop computer, laptop computer, or laboratory computing terminal. In some embodiments, the disclosed systems may be implemented in a server-based environment, such as a centralized server system, application server, database-backed computing environment, research computing node, or institutional computing platform. In some embodiments, the disclosed systems may be implemented in a cloud environment, including one or more virtual machines, containers, serverless execution resources, orchestrated services, or distributed cloud resources. In some embodiments, the disclosed systems may be implemented in a cluster-based or distributed computing environment in which different portions of a simulation are assigned to different nodes, devices, processors, or execution contexts. In some embodiments, the disclosed systems may also be implemented in an accelerated execution environment including one or more vectorized, matrix-based, parallel, or specialized computational resources suitable for carrying out repeated state updates, transform operations, solver operations, propagation operations, or large-scale simulation workloads.

In some embodiments, the disclosed systems may further be deployed in an edge-coupled or instrument-coupled environment. For example, one or more inputs processed by the input module (172) may be received from one or more experimental instruments, sequencers, sensors, image-acquisition systems, assay systems, laboratory information systems, user terminals, external databases, research platforms, or other upstream data sources. In some embodiments, such inputs may include user-specified inputs, experimentally derived inputs, imported files, inferred inputs, preprocessed data, manually defined rule sets, or machine-generated rule sets. In some embodiments, the input module (172) may support one or more input interfaces configured to receive textual inputs, symbolic inputs, structured files, tabular data, machine-readable payloads, application programming interface requests, data-stream inputs, stored datasets, or combinations thereof.

In some embodiments, one or more rules processed by the rule engine (226) may be represented in machine-readable, user-authored, programmatically generated, or transformed formats. For example, one or more rule expressions may be represented in structured text, declarative syntax, condition-action syntax, event-expression syntax, threshold-based syntax, schedule-based syntax, symbolic syntax, domain-specific rule language formats, script formats, or other machine-processable forms. In some embodiments, the input module (172), the rule engine (226), or another associated component may include one or more parsers, validators, translators, compilers, interpreters, tokenizers, normalizers, or transformation engines configured to convert incoming rule expressions into one or more internal executable representations suitable for use during simulation. In some embodiments, one or more parsed rule expressions may be stored in memory, indexed for retrieval, compiled into executable logic, evaluated dynamically at runtime, or modified in response to one or more simulation states, external conditions, or user inputs.

In some embodiments, the RS construct solvers (228) may operate according to different execution paradigms depending on the desired simulation fidelity, computational efficiency, available resources, or implementation preference. For example, one or more simulations may employ exact solutions, approximate solutions, discretized propagators, iterative update schemes, operator-based solutions, transform-based solutions, event-driven updates, fixed-step updates, variable-step updates, deterministic execution, stochastic execution, probabilistic execution, or hybrid execution. In some embodiments, one or more approximate propagation operations may correspond to the approximate propagation step (140), the exponential operator (142), and the evolved state (144), while in other embodiments different propagation techniques may be used. In some embodiments, the time stepper (230) may coordinate fixed interval progression, adaptive interval progression, event-triggered interval progression, branch-dependent interval progression, convergence-driven stopping, or other timing and execution-control strategies. Accordingly, the disclosed computing implementations may support a wide range of execution approaches without requiring any single mathematical solver strategy or deployment form.

In some embodiments, the output module (176) may provide one or more outputs in graphical form, numerical form, textual form, structured form, tabular form, machine-readable form, dashboard form, report form, streamed form, or file-based form. For example, the output module (176) may generate predicted output time-series (232), state-variable output data (234), state trajectories, transformed quantities, rule-activation histories, confidence indicators, exception indicators, comparison reports, simulation summaries, alerts, exported datasets, or downstream integration payloads. In some embodiments, such outputs may be displayed on one or more user interfaces, transmitted to one or more external systems, stored in one or more local or remote storage resources, written to one or more databases, included in one or more dashboards, exposed through one or more application programming interfaces, or otherwise made available for downstream analysis, control, visualization, validation, intervention design, or reporting. In some embodiments, intermediate states, final states, output histories, metadata, and audit records may be retained for replay, traceability, reproducibility, comparison, or later reprocessing.

In some embodiments, communication among the input module (172), the dynamic simulation engine (174), the output module (176), the state manager (224), the rule engine (226), the RS construct solvers (228), and the time stepper (230) may occur through direct function calls, shared memory access, inter-process communication, message queues, event buses, service calls, remote procedure calls, application programming interfaces, data streams, or combinations thereof. In some embodiments, one or more such components may operate synchronously, asynchronously, in batch mode, in streaming mode, in interactive mode, or in hybrid execution mode. In some embodiments, one or more execution instances may be replicated for load balancing, fault tolerance, high-throughput simulation, scenario branching, comparative analysis, or parallel exploration of multiple candidate initial states or rule sets.

In some embodiments, security, access control, integrity management, version control, logging, auditability, and reproducibility features may also be incorporated into the disclosed implementations. For example, one or more rule sets, solver settings, state histories, output histories, and simulation configurations may be versioned, tracked, time-stamped, access-restricted, or cryptographically protected according to implementation preference. In some embodiments, one or more outputs may be associated with provenance information identifying the input conditions, rule definitions, execution settings, and computational environment under which the outputs were generated. Such features may be useful in research, clinical, laboratory, industrial, or regulatory settings in which reproducibility, traceability, or controlled access may be desired.

In some embodiments, one or more aspects of the disclosed systems and methods may be embodied in instructions stored on one or more non-transitory computer-readable media. Such instructions, when executed by one or more processors, may cause the one or more processors to perform any one or more of the operations described herein, including receiving an initial DNARP state, receiving one or more evolution rules, maintaining one or more dynamic state representations, evaluating one or more Recognition Physics (RS)-based rules, applying one or more governing constructs, propagating one or more biological states over time, generating one or more predicted outputs, storing one or more output data products, and providing one or more results to a user or external system. In some embodiments, the non-transitory computer-readable media may include one or more magnetic media, optical media, solid-state storage media, semiconductor memories, persistent storage devices, distributed storage resources, or combinations thereof.

In some embodiments, the disclosed computing and deployment implementations may therefore provide a flexible technical foundation for carrying out the Recognition Physics (RS)-based dynamic DNARP simulations described herein across a wide variety of hardware environments, software architectures, deployment contexts, and execution models. Accordingly, the systems and methods described herein should not be understood as limited to any particular processor type, memory arrangement, module partition, interface format, rule-representation format, solver implementation, timing strategy, deployment topology, or storage medium, unless expressly stated otherwise.

What is claimed is:

1. A computer-implemented method for simulating dynamics of a biological process involving a DNA molecule, the method comprising:
defining, in a computer memory, an initial DNA Recognition Physics state D(t0)=(S(t0), H(t0), E(t0)), wherein S is a sequence component, H is a shape component, and E is an energy component;
defining, in the computer memory, one or more Recognition Physics (RS)-based evolution rules specifying how at least one of the sequence component, the shape component, or the energy component changes over time in response to one or more simulated biological interactions or stimuli;
evolving, using one or more processors, the state D(t) over a time interval by iteratively applying one or more time-dependent governing constructs comprising a DNA Lagrangian LDNA(t), a DNA operator H\hat{ }DNA(t), and a DNA Recognition Transform FDNA(E(t)); and
generating, by an output module, a predicted time course of one or more functional outputs derived from the evolved state D(t).

2. The method of claim 1, wherein the state D(t) is evolved using one or more RS-derived constants without empirical fitting of rate constants for the biological process.

3. The method of claim 1, wherein the one or more evolution rules comprise a rule that modifies sequence accessibility, shape, or energy in response to a binding event, a regulatory interaction, an excitation event, a relaxation event, or a conformational change.

4. The method of claim 1, wherein evolving the state D(t) comprises solving a time-dependent Schrödinger-like equation using H\hat{ }DNA(t) or applying a propagation step based on H\hat{ }DNA(t) over one or more time steps.

5. The method of claim 1, wherein generating the predicted time course of the one or more functional outputs comprises determining a gene expression rate, a coherence level, a regulatory activity, or a protein concentration from the evolved state D(t).

6. The method of claim 1, wherein the biological process is selected from the group consisting of gene regulation, response to drug exposure, response to environmental stress, circadian rhythm dynamics, and DNA repair.

7. A computational system for simulating dynamics of a biological process involving a DNA molecule, the system comprising:
one or more processors;
memory communicatively coupled to the one or more processors;
an input module configured to receive an initial DNA Recognition Physics state D(t0)=(S(t0), H(t0), E(t0)) and one or more Recognition Physics (RS)-based evolution rules;
a dynamic simulation engine executable by the one or more processors and configured to apply the one or more evolution rules to update components of D(t) over successive time intervals and to apply one or more time-dependent governing constructs comprising LDNA(t), H\hat{ }DNA(t), and FDNA(E(t)) to determine state evolution and one or more functional outputs; and
an output module configured to provide a predicted time course of the one or more functional outputs derived from the evolved state D(t).

8. The system of claim 7, wherein the output module is configured to provide time-series data representing one or more of sequence accessibility, shape, energy, gene expression rate, coherence level, or regulatory activity.

9. The system of claim 7, wherein the dynamic simulation engine comprises a state manager configured to maintain a current DNA Recognition Physics state D(t), a rule engine configured to evaluate and apply the one or more RS-based evolution rules to the current DNA Recognition Physics state D(t), one or more Recognition Physics construct solvers configured to apply one or more of the DNA Lagrangian LDNA(t), the DNA operator H\hat{ }DNA(t), and the DNA Recognition Transform FDNA(E(t)) to the current DNA Recognition Physics state D(t), and a time stepper configured to advance the current DNA Recognition Physics state D(t) across successive simulation intervals.

10. The system of claim 9, wherein the input module is further configured to validate the initial DNA Recognition Physics state D(t0) and to parse the one or more RS-based evolution rules into one or more machine-processable internal representations for use by the dynamic simulation engine.

11. The system of claim 9, wherein the state manager is configured to store at least one of a current state, a prior state, an intermediate state, or a checkpoint state associated with the simulation.

12. The system of claim 9, wherein the rule engine is configured to evaluate at least one event-triggered rule, threshold-triggered rule, scheduled rule, deterministic rule, probabilistic rule, or hybrid rule.

13. The system of claim 9, wherein the time stepper is configured to manage fixed simulation intervals, variable simulation intervals, adaptive simulation intervals, or event-driven simulation intervals.

14. The system of claim 9, wherein the output module is configured to generate predicted output time-series and state-variable output data associated with the evolved DNA Recognition Physics state D(t).

15. A non-transitory computer-readable medium storing instructions that, when executed by one or more processors, cause the one or more processors to:
receive an initial DNA Recognition Physics state D(t0)=(S(t0), H(t0), E(t0)) and one or more RS-based evolution rules;
validate the initial DNA Recognition Physics state D(t0) and parse the one or more RS-based evolution rules into one or more machine-processable internal representations;
maintain a current DNA Recognition Physics state D(t);
evaluate and apply the one or more RS-based evolution rules to update at least one of S(t), H(t), or E(t);
apply one or more governing constructs comprising LDNA(t), H\hat{ }DNA(t), and FDNA(E(t)) to propagate the current DNA Recognition Physics state D(t) across one or more simulation intervals; and
generate predicted output time-series and state-variable output data derived from the propagated DNA Recognition Physics state D(t).

16. The non-transitory computer-readable medium of claim 15, wherein parsing the one or more RS-based evolution rules comprises converting textual, symbolic, structured, graphical, machine-readable, or user-defined input into the one or more machine-processable internal representations.

17. The non-transitory computer-readable medium of claim 15, wherein maintaining the current DNA Recognition Physics state D(t) comprises storing time-indexed values corresponding to at least one of sequence accessibility, structural or conformational state, energy or coherence state, or a derived transform value.

18. The non-transitory computer-readable medium of claim 15, wherein applying the one or more governing constructs comprises computing an exact solution, an approximate solution, a discretized solution, a propagator-based solution, an iterative solution, or a hybrid solution.

19. The non-transitory computer-readable medium of claim 15, wherein generating the predicted output time-series and the state-variable output data comprises generating time-indexed values corresponding to at least one predicted functional output and at least one state trajectory of the propagated DNA Recognition Physics state D(t).

20. The non-transitory computer-readable medium of claim 15, wherein a value generated during one simulation interval is used as an input, a condition, or a trigger for evaluation of at least one Recognition Physics (RS)-based evolution rule during a later simulation interval.
